(19) United States
(12) Patent Application Publication
(10) Pub. No.: US 2026/0315043 A1
(43) Pub. Date: Oct. 15, 2026

(54) SAMPLE COLLECTION KIT

(71) Applicant: MAWI DNA TECHNOLOGIES LLC, Hayward, CA (US)

(72) Inventor: Bassam El-Fahmawi, Hayward, CA (US)

(21) Appl. No.: 19/107,511

(22) PCT Filed: Aug. 29, 2023
(86) PCT No.: PCT/US2023/073110

§ 371 Date: Feb. 27, 2025

Related U.S. Application Data
(60) Provisional application No. 63/402,387, filed on Aug. 30, 2022.

(51) Int. Cl. A61B 10/00

(52) U.S. Cl. CPC A61B 10/0045

(57) ABSTRACT

A storage container for storing a sample collection device including a body defining a first storage volume and a first opening through which the first storage volume may be accessed, a cover coupled to the body adjustable between a closed position, in which the cover restricts access to the first storage volume via the first opening, and an open position, in which the cover does not restrict access to the first storage volume via the first opening, a first securing element coupled to the cover and configured to retain the cover in the closed position, and a second securing element coupled to the cover and configured to retain the cover in the closed position independent of the first securing element, where the second securing element is a one-time use device, where the second securing device interacts with the cover at a first point, and where the first point can be detached from the remainder of the cover.

### CROSS-REFERENCE TO RELATED APPLICATION(S)

This application claims priority to U.S. Provisional Patent Application No. 63/402,387, filed on Aug. 30, 2022, the entire contents of which are fully incorporated herein by reference.

### FIELD OF THE INVENTION

The present disclosure relates to a sample collection kit, and more specifically an improved sample collection kit including the materials needed to securely track and ship a biological sample.

### BACKGROUND

For in-home biological sample collection, it is typically needed to ship or otherwise convey the materials for sample collection to the patient and then return the collected sample to a lab or other facility for review and analysis.

### SUMMARY

In one aspect, the disclosure provides a storage container for storing a sample collection device, the storage container including a body defining a first storage volume, where the first storage volume includes a first opening through which the first storage volume may be accessed, a cover coupled to the body, wherein the cover is adjustable between a closed position, in which the cover restricts access to the first storage volume via the first opening, and an open position, in which the cover does not restrict access to the first storage volume via the first opening, a first securing element coupled to the cover and configured to retain the cover in the closed position, and a second securing element coupled to the cover and configured to retain the cover in the closed position independent of the first securing element, wherein the second securing element is a one-time use device, wherein the second securing device interacts with the cover at a first point, and where the first point can be detached from the remainder of the cover.

Alternatively or additionally, in any combination, where at least one of the first securing element and the second securing element include adhesive.

Alternatively or additionally, in any combination, where the first securing element is a one-time use device.

Alternatively or additionally, in any combination, where the cover is integrally formed with the body.

Alternatively or additionally, in any combination, where the cover includes a first portion configured to selectively obstruct access to the first storage volume via the first opening, a second portion extending from the first portion, where the first securing element interacts with the cover via the second portion, and a third portion extending from the second portion opposite the first portion, where the second securing element interacts with the cover via the third portion.

Alternatively or additionally, in any combination, where the third portion is detachable from the second portion.

Alternatively or additionally, in any combination, where the third portion is connected to the second portion via a perforated connector.

Alternatively or additionally, in any combination, where the first storage volume is sub-divided by a wall into a first storage volume portion and a second storage volume portion, and where the wall is movable between a first position, in which the first storage volume portion is accessible via the first opening and the second storage volume portion is not accessible via the first opening, and a second position, in which both the first storage volume portion and the second storage volume portion are accessible via the first opening.

Alternatively or additionally, in any combination, where the wall defines an aperture sized to receive at least a portion of the sample collection device therein.

In another aspect, the disclosure provides a storage container including a body defining a storage volume, where the storage volume includes a first opening through which the first storage volume may be accessed, a cover, where the cover is adjustable between a closed position, in which the cover restricts access to the first storage volume via the first opening, and an open position, in which the cover does not restrict access to the first storage volume via the first opening, where the cover includes a first portion, and a second portion extending from the first portion, a first securing element coupled to the first portion of the cover and configured to retain the cover in the closed position, and a second securing element coupled to the second portion of the cover and configured to retain the cover in the closed position independent of the first securing element.

Alternatively or additionally, in any combination, where the second securing member is a one-time use device.

Alternatively or additionally, in any combination, where the second securing member is an adhesive.

Alternatively or additionally, in any combination, where the cover is formed integrally with the body.

Alternatively or additionally, in any combination, where the cover further includes a third portion adjustably coupled to the body and configured to at least partially obstruct the first opening, and wherein the first portion extends from the third portion.

Alternatively or additionally, in any combination, where the second portion is detachable from the first portion.

Alternatively or additionally, in any combination, where the second portion is coupled to the first portion by a perforated strip.

In another aspect, the disclosure provides a method of transporting and collecting a secure sample in a sample collection device, the method including providing a storage container defining a first storage volume accessible via a first opening, the storage container including a cover adjustable between a closed position, in which the first storage volume is not accessible via the first opening, and an open position, in which the first storage volume is accessible via the first opening, the storage container also including a first securing element and a second securing element, placing an empty sample collection device into the first storage volume via the first opening, securing the cover in the closed position with the first securing element, opening the storage container by detaching the portion of the cover interacting with the first securing element and placing the cover in the open position, removing the empty sample collection device from the first storage volume and filling it with a sample, returning the filled sample collection device to the first storage volume, securing the cover in the closed position with the second securing element.

Alternatively or additionally, in any combination, where at least one of the first securing element and the second securing element are a one-time use device.

In another aspect, the disclosure provides, a sample collection device including a body defining a sample collection volume therein, where at least a portion of the body is transparent, and indicia visible along a sight line that passes through the sample collection volume.

Alternatively or additionally, in any combination, further comprising a pre-determined volume of buffer in the sample collection volume, and where the sight line passes through the buffer when the sample collection device is in an upright orientation.

Alternatively or additionally, in any combination, where the buffer has a first state where no sample is dissolved therein, where the buffer has a first opacity in the first state, where the buffer has a second state where desired volume of sample is dissolved therein, where the buffer has a second opacity greater than the first opacity in the second state.

Alternatively or additionally, in any combination, where the sight line defines a sight length, and where the sight length is such that the indicia is not visible when the buffer is in the second state.

Alternatively or additionally, in any combination, further comprising a label applied to the exterior of the body, where the label has a first side facing away from the body and a second side opposite the first side facing toward the body, and where the indicia is formed on the second side.

Alternatively or additionally, in any combination, where the indicia is printed onto the second side of the label.

Alternatively or additionally, in any combination, where the indicia includes black symbol printed on a white background.

Alternatively or additionally, in any combination, where the label is applied to the exterior of the body to produce a covered portion and a window portion.

Alternatively or additionally, in any combination, where the sight line passes through the window portion.

In another aspect, the disclosure provides, a sample collection device including, a body defining a sample collection volume therein, where at least a portion of the body is transparent, and a label applied to the exterior of the body, the label having an exterior surface facing away from the body and an interior surface facing toward the body, and indicia applied to the interior surface of the label.

Alternatively or additionally, in any combination, where the indicia is visible through the body.

Alternatively or additionally, in any combination, further comprising a pre-determined volume of buffer positioned within the sample collection volume.

Alternatively or additionally, in any combination, where the indicia is visible through the buffer.

Alternatively or additionally, in any combination, where the label extends a portion of the distance around the perimeter to define a gap, and where the indicia is at least partially aligned with the gap.

Alternatively or additionally, in any combination, where the indicia is opposite the gap.

In another aspect, the disclosure provides, a kit for collecting and storing a biological sample, the kit including a sample collection device defining a sample collection volume and a first open end, a cap configured to be removably coupled to the first open end to enclose the sample collection volume, a swab enclosed in first package, a storage container sized to receive the sample collection device, the cap, and the swab therein, and a set of labels configured to be applied to at least one of the sample collection device, the cap, the first package, the swab, and the storage container, and where each label of the set of labels contains matching identification information thereon.

Alternatively or additionally, in any combination, further comprising a funnel having a first end configured to be removably coupled to the first open end of the sample collection device and a second end opposite the first end, and where the second end is larger than the first end.

Alternatively or additionally, in any combination, where each label of the set of labels has a first surface upon which the identifying information is applied, and a second surface opposite the first surface where an adhesive is applied.

Alternatively or additionally, in any combination, where at least one label of the set of labels further includes secondary indicia applied to the second side.

Alternatively or additionally, in any combination, where the at least one label is applied to the sample collection device.

Alternatively or additionally, in any combination, where the identifying information may include any one of a QR code, a bar code, and alpha-numeric text.

Alternatively or additionally, in any combination, where the storage container defines a storage volume accessible via a first inlet, where the storage container includes a barrier at least partially positioned within the storage volume and configured to sub-divide the storage volume into a first volume portion and a second volume portion, and where the second volume portion is sized to receive the swab therein.

Alternatively or additionally, in any combination, where the barrier is adjustable between a first position, in which the first volume portion is accessible via the inlet and the second volume portion is not accessible via the inlet, and a second position, in which both the first volume portion and the second volume portion are accessible via the inlet.

Alternatively or additionally, in any combination, where the barrier defines an aperture therein sized to receive at least a portion of the sample collection device therein.

Alternatively or additionally, in any combination, where the aperture is sized and shaped so that the open end of the sample collection device is accessible via the inlet when the sample collection device is positioned within the aperture.

In another aspect, the disclosure provides, a kit for collecting and storing a biological sample, the kit including a sample collection device defining a sample collection volume and a first open end, where the first sample collection device has a first indicia, a cap configured to be removably coupled to the first open end to enclose the sample collection volume, where the cap includes a second indicia, a swab enclosed in first package, and where one of the swab and the first package includes a third indicia, a storage container sized to receive at least one of the sample collection device, the cap, and the swab therein, and where the storage container includes a fourth indicia, and where the first indicia, the second indicia, the third indicia, and the fourth indicia all include matching identification information.

Alternatively or additionally, in any combination, where each indicia includes a sticker applied to its respective element.

Alternatively or additionally, in any combination, where the storage container defines a storage volume accessible via an inlet, and where the storage volume is sized to receive the sample collection device, the cap, and the swab therein.

Alternatively or additionally, in any combination, where the storage container further includes a barrier configured to separate the storage volume into a first portion and a second portion, and where the second portion is sized to receive the swab therein.

Alternatively or additionally, in any combination, where the barrier defines an aperture sized to at least partially receive the sample collection device therein, and where storage container is configured such that the first open end of the sample collection device is positioned within the first portion when the sample collection device is positioned within the aperture.

Alternatively or additionally, in any combination, where the barrier is adjustable between a first position, in which the first portion is accessible via the inlet and the second portion is not accessible via the inlet, and a second position, in which both the first portion and the second portion are accessible via the inlet.

Alternatively or additionally, in any combination, where the barrier defines an aperture sized to at least partially receive the sample collection device therein, and where storage container is configured such that the first open end of the sample collection device is positioned within the first portion when the sample collection device is positioned within the aperture.

Alternatively or additionally, in any combination, further comprising a funnel having a first end configured to be removably coupled to the first open end of the sample collection device and a second end opposite the first end, and where the second end is larger than the first end.

Alternatively or additionally, in any combination, where the first open end of the sample collection device includes external threads formed thereon.

Other aspects of the disclosure will become apparent by consideration of the detailed description and accompanying drawings.

### BRIEF DESCRIPTION OF THE DRAWINGS

FIG. 1 illustrates a sample collection kit.

FIG. 2 is a front view of a container of the sample collection kit of FIG. 1.

FIG. 3 is a top perspective view of the container of FIG. 2 with the cover and the partition in open configurations.

FIG. 4 is a top perspective view of the container of FIG. 2 with the cover in an open configuration and the partition in a closed configuration.

FIG. 5 is a top perspective view of the container of FIG. 2 with a sample collection device stored therein.

FIG. 6 is a top perspective view of the container of FIG. 2 with the cover in a first secured closed condition.

FIG. 7 is a top perspective view of the container of FIG. 2 with the cover in an open condition.

FIG. 8 is a top perspective view of the container of FIG. 2 with the cover in a second secured closed condition.

FIGS. 9 and 10 illustrate alternative embodiments of the container.

FIG. 11 illustrates a piece of sheet material.

FIG. 12 is a top perspective view of the sample collection device of the kit of FIG. 1.

FIG. 13 illustrates the sample collection device of FIG. 12 with the cover removed.

FIG. 14 is a side view of the sample collection device of FIG. 13.

FIG. 15 is a section view taken along line 15-15 of FIG. 14.

FIG. 16 is a side view of the sample collection device of FIG. 12 with a funnel attached thereto.

FIG. 17 illustrates identification elements of the kit of FIG. 1.

### DETAILED DESCRIPTION

Before any embodiments of the disclosure are explained in detail, it is to be understood that the disclosure is not limited in its application to the details of the formation and arrangement of components set forth in the following description or illustrated in the accompanying drawings. The disclosure is capable of supporting other implementations and of being practiced or of being carried out in various ways.

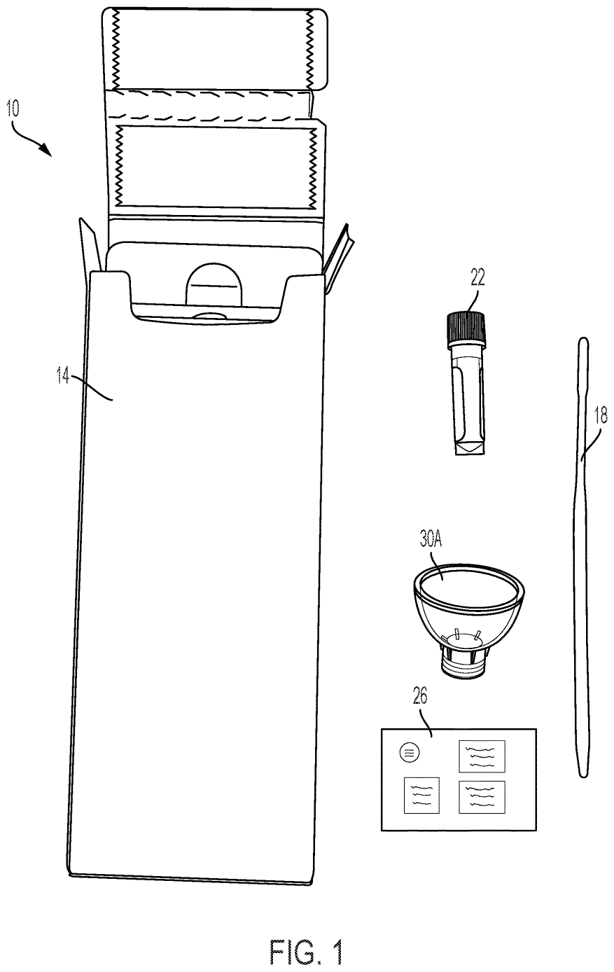

FIG. 1 illustrates a sample collection kit 10 containing materials needed to collect and store an in-home biological sample. More specifically, the sample collection kit 10 includes materials needed to securely track and ship an un-used kit to a pre-determined patient, allow the patient to access the materials needed to collect and store the desired sample, and includes the materials needed to then securely track and ship the collected sample back to the lab for analysis. In the illustrated embodiment, the kit 10 includes a container 14, a swab 18, a sample collection device 22, and identification elements 26. While not shown, additional materials may also be included in the kit 10 such as, but not limited to, various collection accessories 30 (discussed below), instructions (not shown), branding indicia, postage indicia, holders or supports, temperature control elements (e.g., in instances where the sample must be kept at a pre-determined temperature), and the like.

The container 14 of the kit 10 includes a body 36 defining a storage volume 42 that is sized to receive at least the sample collection device 22 therein. During use, the container 14 is configured to restrict access to the volume 42, placing the volume 42 in a secure closed state on multiple, independently verifiable occasions. For the purposes of this application, the storage volume 42 is considered in a secure closed state when the storage volume 42 is enclosed such that access to the volume 42 can only reasonably be obtained by taking steps that will either irreversibly damage, modify, and/or mark the container 14 in such a way so as to be easily identified by the user. In the illustrated embodiment, the container 14 is configured for two-way secured travel whereby the storage volume 42 can be placed in a first secured state for travel to the patient, opened to gain access to the storage volume 42 to remove and use any materials contained therein, placed in a second secured state for travel back to the lab, and then opened a second time to gain access to the storage volume 42 to remove the materials contained therein. While the illustrated container 14 is designed for two-way travel, it is understood that in other embodiments the container 14 may be configured to be placed in a secure state more than two times (e.g., in instances where the sample needs to be securely shipped to multiple locations for analysis and/or cataloging).

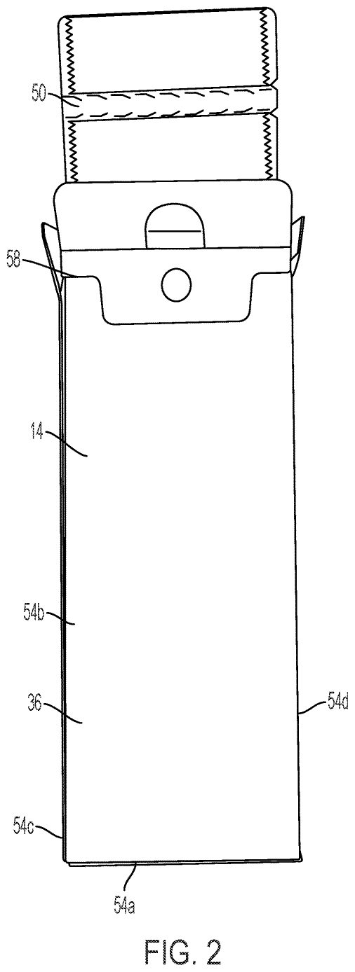

As shown in FIGS. 2-8, the container 14 includes an exterior body or shell 36 at least partially enclosing the storage volume 42 therein, an aperture or opening 46 formed by the body 36 to provide access to the storage volume 42, and a cover 50 configured to selectively restrict access to the volume 42 via the opening 46. More specifically, the body 36 of the container 14 includes one or more walls 54 that at least partially enclose the volume 42 and define the opening 46. More specifically, the body 36 is rectangular in shape having a base wall 54a, and one or more side walls 54b, 54c, 54d, 54e extending from the base wall 54a to produce a first open end 58 opposite the base wall 54a.

The container 14 also includes an opening 46 defined by the body 36 through which the user can access the storage volume 42 from the outside. The opening 46 is sized and shaped to permit the sample collection device 22 and other elements to pass therethrough and be removed from or inserted into the volume 42 during use. In the illustrated embodiment, first open end 58 of the body 36 forms the opening 46; however, in other embodiments the opening 46 may be placed in other locations as needed. In still other embodiments, more than one opening each having its own cover 50 (described below) may be present. In still other embodiments,

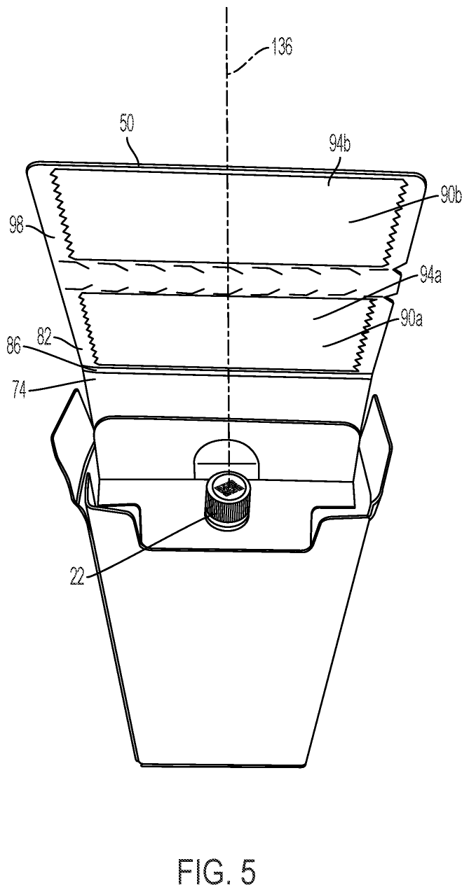

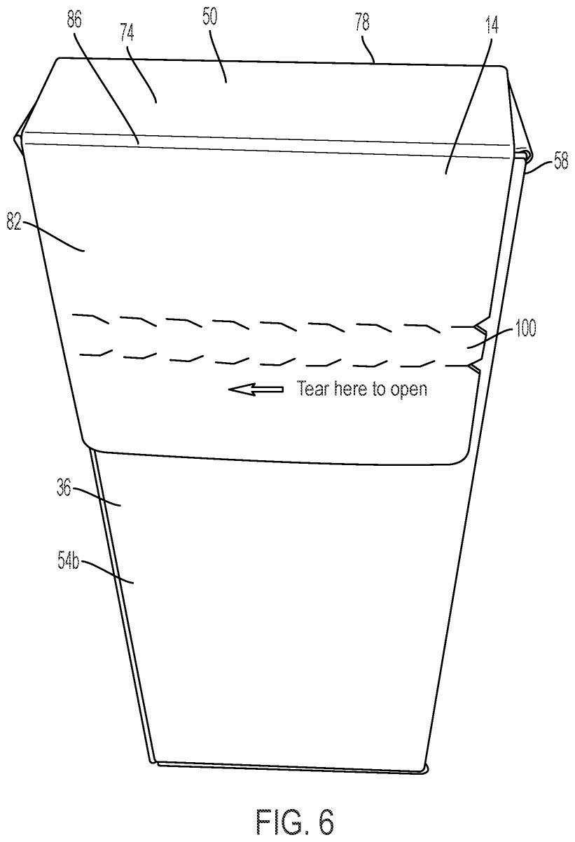

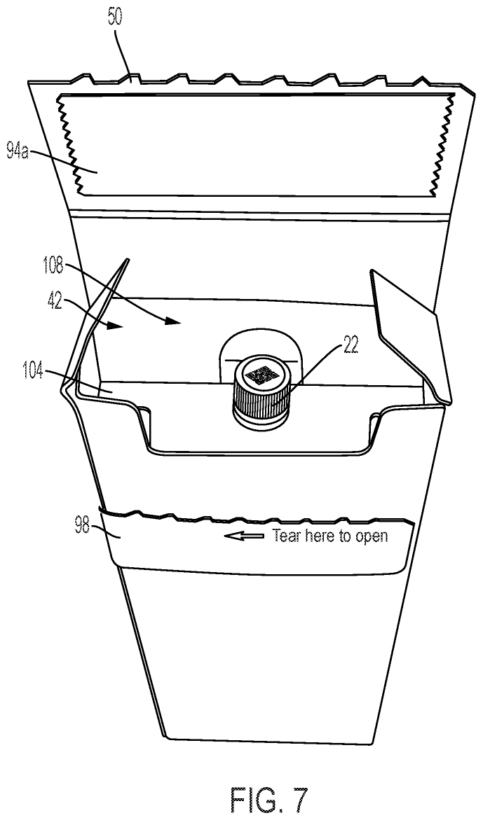

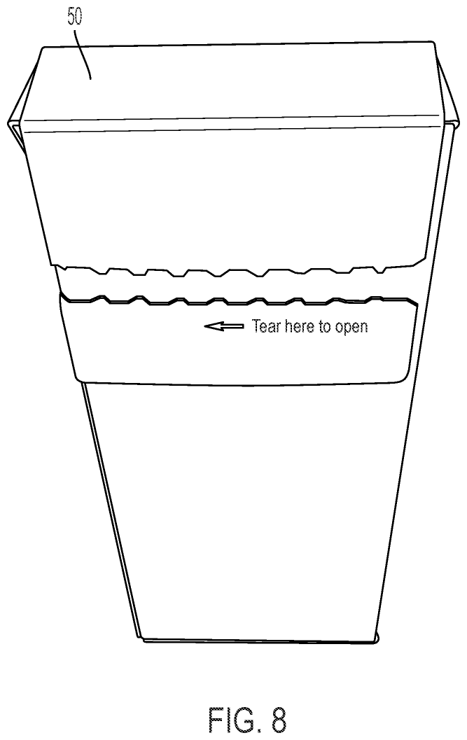

The cover 50 of the container 14 is sized and shaped to at least partially cover the opening 46 and selectively restrict access to the volume 42. During use, the cover 50 is movable with respect to the body 36 between a closed position (see FIGS. 6 and 8), in which the cover 50 restricts access to the volume 42 via the opening 46, and an open position (see FIGS. 5 and 7), in which the cover 50 does not restrict access to the volume 42 via the opening 46. While the illustrated cover 50 is configured to restrict access to the volume 42 when in the closed position, it is understood that in other embodiments the cover 50 may also hermetically seal the volume 42 to maintain a sterile environment therein.

In the illustrated embodiment, the cover 50 is formed integrally with the body 36 having a first panel 74 extending from the second open end of a sidewall 54c to form a first hinge 78 therebetween, and a second panel 82 extending from the first panel 74 opposite the first hinge 78 to form a second hinge 86 therebetween. Together, the first panel 74, second panel 82, and two hinges 78, 86 are configured so that the cover 50 may be pivoted relative to the body 36 between the open and closed positions (see FIGS. 5-8). More specifically, when the cover 50 is in the closed position, the cover 50 is sized and shaped such that the first panel 74 encloses the first open end 58 of the body 36 while the second panel 82 extends along and lies against a corresponding side wall 54b of the body 36. While the illustrated cover 50 is shown being formed integrally with the body 36 and pivotable relative thereto, in other embodiments the cover 50 may be formed as a separate unit and attached thereto. In still other embodiments, the cover 50 may be threaded onto the body 36 and secured by a coupler 90.

As shown in FIG. 5, the container 14 also includes a plurality of one-time use couplers 90. Each coupler 90, in turn, is configured to independently retain the cover 50 in the closed position whereby, once closed, the cover 50 can only reasonably be opened by taking steps that will either damage, modify, and/or mark the cover 14 in such a way so as to be easily identified by the user (e.g., a secured closed position). More specifically, the couplers 90 are configured so that each coupler 90a, 90b can independently retain the cover 50 in a secured closed condition. As such, the container 14 is capable of being placed in a secured closed condition on multiple separate occasions (e.g., once for each coupler 90 that is present).

With continued reference to FIG. 5, the first coupler 90a includes a one-time use connector or securing element 94a attached to the cover 50. In the illustrated embodiment, the connector 94a includes an adhesive strip that is attached directly to the second panel 82 of the cover 50. During use, the adhesive strip is generally adjusted between a disengaged configuration, in which a layer of adhesive backing is attached to the strip such that it will not couple to the body 36, and an engaged configuration, in which the adhesive backing has been removed and the strip will couple to the body 36 when placed in contact therewith. When the coupler 90a is in the activated configuration, the user may place the cover 50 in the closed position whereby the connector 94 will couple to and secure the second panel 82 of the cover 50 against the side wall 54b—thereby retaining the cover 50 in a secured closed condition.

While the illustrated connector 94a is an adhesive strip, it is understood that in other embodiments other forms of one-time-use connectors may be used. For example, the securing element 94a may include, but is not limited to, clips, ratchets, stickers, seals, tabs, wire, tamper resistant materials, and the like. Furthermore, while the first coupler 90a of the illustrated construction is shown being integrated into the second panel 82 of the cover 50, it is understood that in other embodiments the first coupler 90a may also be formed separately from the cover 50. In such embodiments, the first coupler 90a may be incorporated into a separate tab (described below), attached separately as a seal, and the like.

As shown in FIGS. 5 and 6, the second coupler 90b includes a tab 98 removably attached to the cover 50 by way of a one-time use releasable joint 100, and a one-time use connector or securing element 94b positioned on the tab 98. More specifically, the tab 98 extends from the second panel 82 of the cover 50 opposite the second hinge 86. In the illustrated embodiment, the connector 94b includes an adhesive strip that is attached directly to the tab 98. During use, the adhesive strip is generally adjusted between a disengaged configuration, in which a layer of adhesive backing is attached to the strip such that it will not couple to the body 36, and an engaged configuration, in which the adhesive backing has been removed and the strip will couple to the body 36 when placed in contact therewith. When the coupler 90b is in the activated configuration, the user may place the cover 50 in a closed position whereby the connector 94b will couple to and secure the tab 98 against the side wall 54b—thereby retaining the cover 50 in a secured closed condition.

While the illustrated connector 94b is an adhesive strip, it is understood that in other embodiments other forms of one-time-use connectors may be used. For example, the securing element 94b may include, but is not limited to, clips, ratchets, stickers, seals, tabs, wire, tamper resistant materials, and the like. Furthermore, while the second coupler 90b of the illustrated construction is shown being integrated into the tab 98, it is understood that in other embodiments the second coupler 90b may also be formed separately from the tab 98. In such embodiments, the second coupler 90b may be attached separately as a seal, and the like.

The releasable joint 100 between the tab 98 and the cover 50 is a one-time releasable joint such that once the tab 98 is detached from the cover 50 it is not capable of being reasonably re-attached using the joint 100. In the illustrated embodiment, the releasable joint 100 includes a perforated strip of material that can be torn away by the user to separate the tab 98 from the cover 50. However, in other embodiments, different forms of releasable joint 100 may be used such as but not limited to a cutting string, indicia indicating a line to be cut, a break-away seal, and the like.

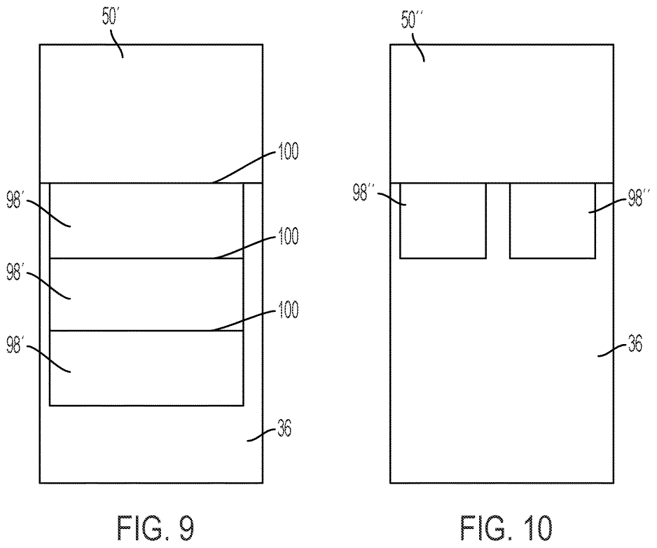

In the illustrated embodiment, the container 14 includes two couplers 90a, 90b, each of which incorporate a one-time use connector 94a, 94b therein such that the cover 50 may be placed in a secured closed condition on two separate occasions. However, in other embodiments more couplers 90 may be present to allow the cover 50 to be placed in a secured closed position on more than two occasions. For example, in some embodiments the container 14 may include two or more tabs 98′ arranged in series and extending from the cover 50—each having a corresponding one-time use connector 94 thereon and a one-time use releasable joint 100 therebetween (see FIG. 9). In still other embodiments, the container 14 may include two or more tabs 98″ arranged in parallel and extending from the cover 50—each having a corresponding one-time use connector 94 therein (See FIG. 10). In still other embodiments, one or more of the couplers 90 may include a multi-use connector 94 thereon (e.g., velcro, re-usable adhesive, bendable tabs, and the like) to allow that particular connector 94 to be used for multiple, non-tamper resistant openings and closings of the volume 42 using a single coupler 90. In still other embodiments, a combination of multi-use and one-time use couplers 90 may be present.

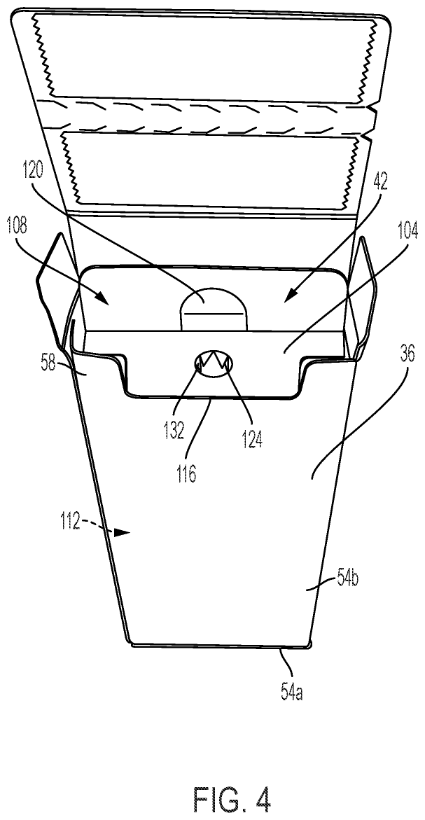

As shown in FIG. 4, the container 14 also includes a shelf or partition 104 configured to subdivide the volume 42 into a first portion 108 and a second portion 112. More specifically, the partition 104 includes a planar wall positioned between the base wall 54a and the open end 58 that subdivides the volume 42 into the first portion 108, positioned between the partition 104 and the open end 58, and the second portion 112 positioned between the partition 104 and the base wall 54a. In the illustrated embodiment, the partition 104 is positioned proximate the open end 58 such that the second portion 112 is larger than the first portion 108. More specifically, the partition 112 is positioned so that the second portion 112 is large enough to receive at least one swab 18 therein. The illustrated partition 104 may also include a handle or tab 120 extending therefrom and allowing the user to manipulate the partition 104 inside the volume 42.

In the illustrated embodiment, the partition 104 is formed integrally with the body 36, being formed by folding the open end 58 of one of the side walls 54b into the volume 42 forming a hinge 116 therebetween. More specifically, the location of the fold (see FIG. 4) generally determines the location of the partition 104 relative to the overall body height.

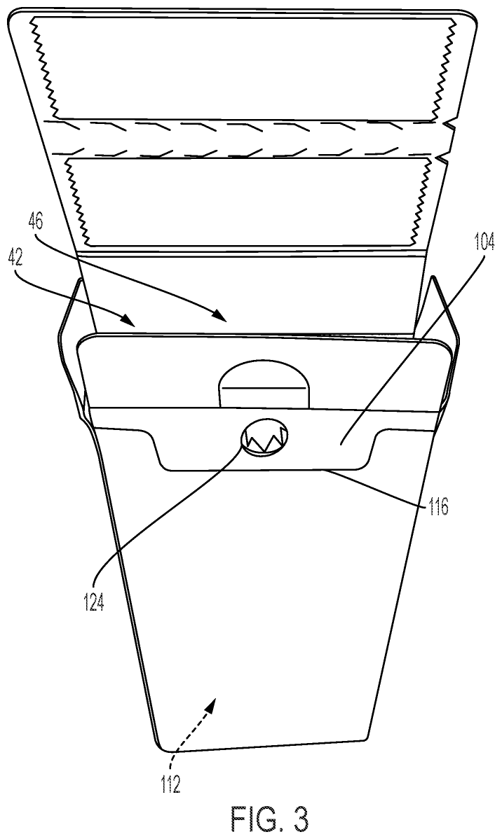

During use, the partition 104 is movable with respect to the body 36 between a closed position (see FIG. 4), in which the partition 104 sub-divides the volume 42 into the first and second portions 108, 112, and an open position (see FIG. 3), in which the partition 104 does not sub-divide the volume 42 into two portions 108, 112. Generally speaking, the partition 104 is generally oriented normal to the sidewalls 54b-e when in the closed position and oriented parallel to the sidewalls 54b-e when in the open position. While the illustrated partition 104 is formed integrally with the body 36, it is understood that in other embodiments the partition 104 may be formed separately from the body 36 and inserted into the volume 42 and coupled to the body 36.

The partition 104 includes a vessel support 124 configured to retain and position the sample collection device 22 within the volume 42 (see FIGS. 4 and 5). More specifically, the vessel support 124 is configured to position the sample collection device 22 within the volume 42 such that the first or open end 128 thereof (described below) is positioned within the first portion 108 of the volume 42 proximate the opening 46. As shown in FIG. 5, the collection device 22 is maintained within the support 124 such that the vessel axis 136 is longitudinally aligned with the body 36. In the illustrated embodiment, the vessel support 124 includes an aperture formed by the partition 104 that includes a plurality of protrusions 132 extending into the aperture to help retain the sample collection device 22 therein during use. While the illustrated vessel support 124 includes an aperture and protrusions, it is understood that in other embodiments different forms of vessel support 124 may be present. For example, the support 124 may include a series of straps, clips, and the like.

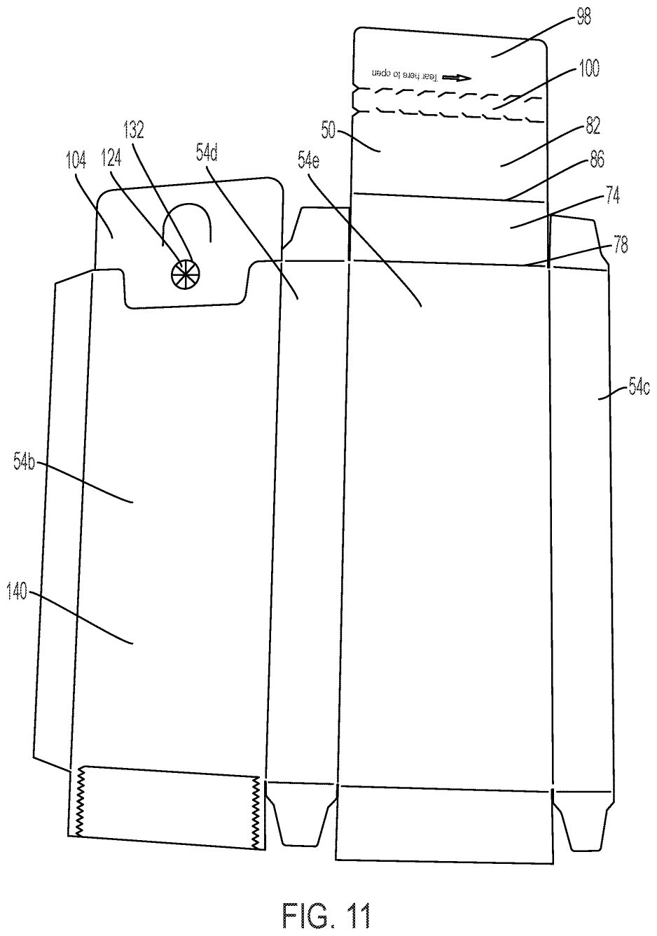

The container 14 as described above is formed from a single continuous piece of sheet material (e.g., cardboard). More specifically, the container 14 is configured so that the body 36, cover 50, and partition 104 can all be created by folding a single piece of planar sheet material in an appropriate manner and applying adhesive where necessary. The illustrated design has a single adhesive joint corresponding with one of the side walls 54c. While the illustrated embodiment is formed from cardboard, it is understood that in other embodiments other forms of sheet material may be used such as, but not limited to, sheet metal, sheet plastic, poster board, and the like. In still other embodiments, the underlying sheet material may be a combination of different types of sheet material. One embodiment of a pre-folded piece of sheet material 140 is shown in FIG. 11.

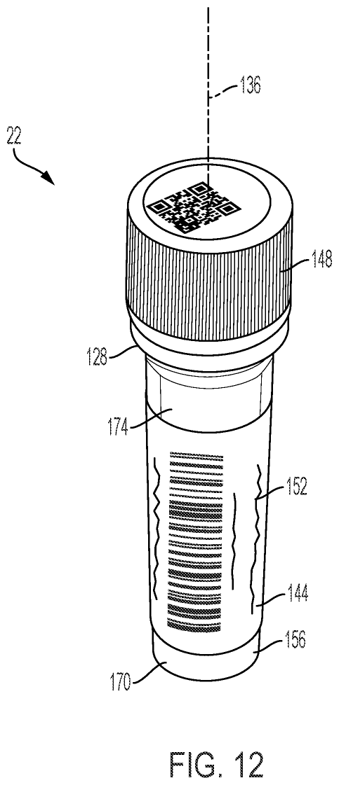

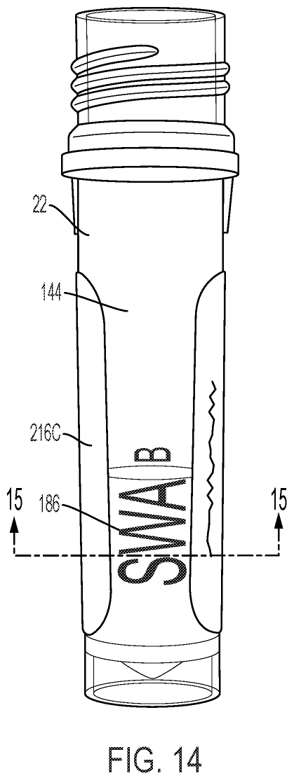

FIGS. 12-16 illustrate one embodiment of the sample collection device 22 configured to receive and retain a biological sample therein. The collection device 22 includes a vessel 144 and a cap or cover 148 removably couplable to the vessel 144. In some embodiments, the vessel 144 may also include a pre-determined volume of buffer fluid or other preserving substance pre-loaded within the vessel 144. The buffer liquid is transparent in its unmixed state (see FIG. 14).

As shown in FIG. 12, the vessel 144 of the collection device 22 includes an elongated body 152 having a first or closed end 156, and a second or open end 128 opposite the first end 156. The vessel 144 also defines a vessel axis 136 and a vessel volume 166 that is accessible via the open end 128. More specifically, the vessel 144 generally includes a base wall 170 and one or more side walls 174 extending from the base wall 170 to define the open end 128. In the illustrated embodiment, the body 152 is substantially cylindrical in shape however in other embodiments, different shapes may be used such as, but not limited to rectangular, polygonal, frusto-conical, and the like.

In the illustrated embodiment, at least a portion of the vessel 144 is formed from transparent material (e.g., clear plastic) such that the user can at least partially see through the at least one side wall 174 (see FIG. 14). More specifically, the illustrated vessel 144 is molded as a single piece of clear plastic. However, in other embodiments portions of the vessel 144 may be formed from both opaque and transparent areas.

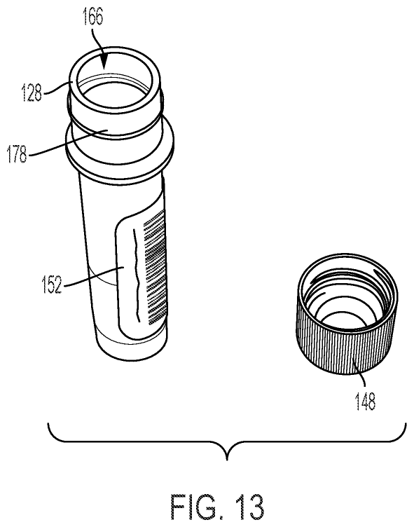

As shown in FIG. 13, the vessel 144 also includes a connection point 178 proximate the open end 128 and configured to allow the cover 148 and/or other accessories (described below) to be removably attached thereto. More specifically, the illustrated connection point 178 includes a set of external threads although in other embodiments different forms of connection may be present such as, but not limited to, a bayonet fit, tabs, lugs, twist-lock, and the like.

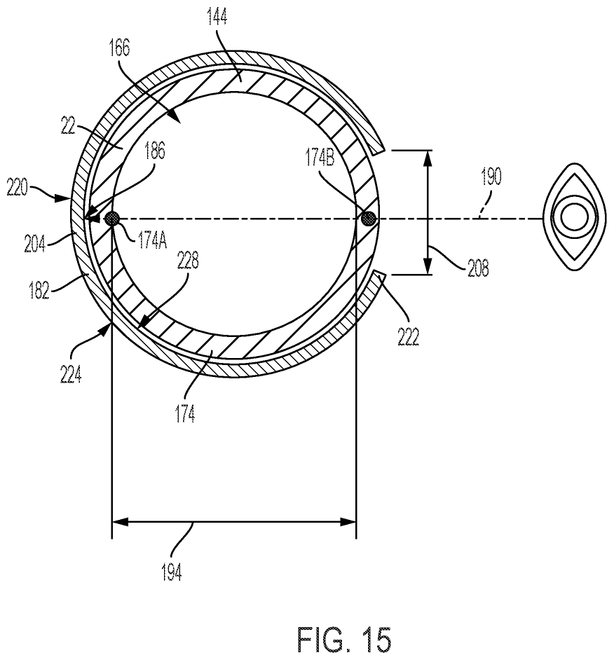

As shown in FIG. 15, the sample collection device 22 also includes a sample verification system 182. The sample verification system 182 serves to provide the user visual feedback regarding the volume of biological sample material contained within the vessel volume 166 at any given time. More specifically, the sample verification system 182 is configured to allow the user to visually identify when the desired volume of biological sample material has been deposited in the volume 166 of the vessel 144.

In the illustrated embodiment, the sample verification system 182 includes indicia 186 attached to or otherwise incorporated into the vessel 144 that can be viewed along a sightline 190 that passes through the vessel volume 166 including any fluid contained therein. The sightline 190, in turn, defines a sight length 194 generally defined as the length of the sightline 190 actually positioned within the volume 166 (see FIG. 15). Together, the relative contrast of the indicia 186 and the sight length 194 generally determine how opaque any fluid contained within the volume 166 must be before the indica 186 becomes obscured and cannot be easily identified by a user looking along the sightline 190. Stated differently, the sample verification system 182 includes indicia 186 visible along a sightline 190 passing through the vessel volume 166 whose visual appearance along the sightline 190 is impacted by the concentration of the sample contained within the vessel volume 166. More specifically, indica 186 having greater contrast (e.g., black letters on a white background) will require a relatively more opaque fluid to become obscured for a given sight length 194 while relatively less contrasted indicia 186 (e.g., dark grey letters on a light grey background) requires relatively less opaque fluid to become obscured for a given sight length 194. Furthermore, a relatively longer the sight length 194 requires a relatively less opaque liquid for the indicia 186 to become obscured while shorter sight length 194 require relatively more opaque liquid for the indicia 186 to become obscured. Using at least these relationships, the manufacturer can tailor the contrast of the indicia 186 and sight length 194 so that the indica 186 becomes obscured at a pre-selected fluid opacity level.

As shown in FIG. 15, the illustrated sample verification system 182 includes indicia 186 that is printed onto the radially inside surface 220 of an opaque label 216C applied to the exterior of the at least one side wall 174 of the vessel 144. As such, the resulting sightline 190 passes through the at least one side wall 174 at a first location 174A, radially through the volume 166 generally oriented perpendicular to the vessel axis 136 (e.g., ±10%), and through the at least one side wall 174 at a second location 174B located opposite the first location 174A. In such an embodiment, the sightline 190 passes through the body 152 twice, requiring the body 152 to be transparent at both the first location 174A and the second location 174B. The resulting layout also generates a sight length 194 generally equal to the diameter of the volume 166.

In the illustrated embodiment, the opaque label 216C is sized to extend around at least a portion of the perimeter of the vessel 144 to produce a gap 208. The gap 208 is positioned opposite the location of the indicia 186 to produce a viewing window 212 through which the sightline 190 passes so the user can view the indicia therethrough. In other embodiments, the opaque label 216C may extend completely around the perimeter of the vessel 144 and include an aperture formed therein to serve as the viewing window 212. In still other embodiments, the label 216C may include one or more transparent regions to serve as the viewing window 212. The illustrated indicia 186 includes black letters printed on a white background for maximum contrast. However, in other embodiments the indicia 186 may include a symbol, logo, pattern, picture, and the like using two or more colors.

In other embodiments, the indicia 186 may be printed on the inside surface of the at least one side wall 174 of the vessel 144 such that the resulting sightline 190 passes through the volume 166 generally perpendicular to the vessel axis 136 (e.g., ±10%), and through the at least one side wall 174 opposite the indicia 186. In such an embodiment, the indicia 186 need only pass through the body 152 of the vessel 144 once. As such, the body 152 need only be transparent in at least that area.

Duringuse, the volume 166 of the vessel 144 begins with a pre-determined volume of buffer solution contained therein such that the sightline 190 passes through not only the volume 166 but the buffer solution contained within the volume 166 when the vessel 144 is in an upright orientation (e.g., the vessel axis 136 is vertical with the open end 128 is located higher than the closed end 156). The buffer solution, in turn, is clear having an opacity of approximately 0 (e.g., ±10%, see FIG. 14) such that the overall opacity of the solution within the volume 166 (e.g., the solution opacity) is initially close to 0 (e.g., ±10%). In such a condition, the indicia 186 is visible by the user.

As the user adds biological material to the vessel 144 via the open end 128 (e.g., via the swab 18, the funnel 30a, and the like), the resulting solution within the volume 166 becomes increasingly more and more opaque (e.g., the solution opacity increases). Stated differently, the solution opacity within the volume 166 has a positive correlation with the ratio of the volume of biological material to the volume of buffer. As such, as biological sample material is added to the volume 166 the solution opacity begins to rise. In the illustrated embodiment, the contrast of the indicia 186 and the sight length 194 are configured so that the indicia 186 becomes obscured when the desired volume of biological sample material is added to the pre-determined volume of buffer (e.g., when the desired ratio of the volume of biological material to the volume of buffer is achieved). Once the patient is no longer able to see the indicia 196, the patient knows a sufficient volume of biological material has been deposited in the vessel 144. Generally speaking, the layout of the sample verification system 182 is configured to accommodate the difference between low sheading and high sheading subjects.

The cover 148 of the sample collection device 22 includes a cylindrical body configured to be releasably attached to the first end 128 of the vessel 144 via the connection point 178 and form a water-tight seal therewith. During use, the cover 148 is adjustable between a closed configuration, in which the volume 166 is completely enclosed and cannot be accessed (see FIG. 12), and an open configuration, in which the volume 166 may be accessed via the open end 128 (see FIG. 13). In the illustrated embodiment, the cover 148 includes a set of internal threads configured to engage the external threads 178 of the vessel 144, however in other embodiments different forms of connection may be used.

The sample collection kit 10 also includes a set of identification elements 26 each configured to allow the individual components of the kit 10 to be associated with a particular biological sample collection. More specifically, the identification elements 26 include a plurality of individual labels 216 (e.g., three labels) that are packaged together and include matching and/or coordinated indicia 220 printed thereon.

In the illustrated embodiment, each label 216 is an adhesive sticker or decal having a first or non-adhesive side 224, and a second or adhesive side 228 (see FIG. 15) opposite the first side 224. Each label 216A-C, in turn, includes indicia 220 printed onto the first side 224 that conveys information necessary to identify and track the biological sample during the collection and testing processes. In the illustrated embodiment, the information may be conveyed using a combination of QR codes, bar codes, alpha-numeric text, logos, diagrams, and the like. As such, by applying the labels 216 to each of the sub-components of the kit 10 (e.g., the container 14, vessel 144, and cover 148), each sub-component can be tracked both individually and with respect to the kit 10 as a whole.

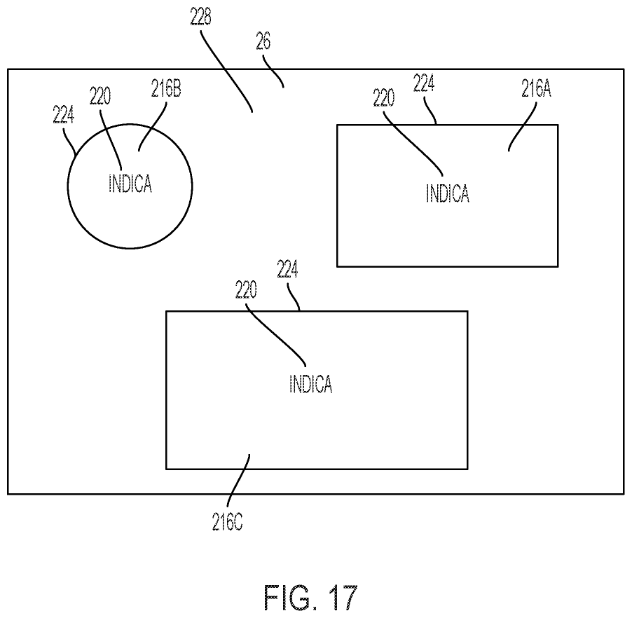

As shown in FIG. 17, the adhesive labels 216A-C are presented on a single backing sheet 228 such that the labels 216A-C can be stored together, as a single unit, in the volume 42 of the container 14 and subsequently applied individually on the sub-components of the kit 10 as a part of the sample collection process. While the illustrated labels 216A-C are stickers and/or decals, it is understood that in other embodiments different forms of identification may be used separately or integrated into the labels 216A-216C such as, but not limited to, seals, printed indicia directly on the element itself, RFID tags, and the like.

In the illustrated embodiment, the plurality of labels 216A-C include a container label 216A, a cap label 216B, and a vessel label 216C. The container label 216A and cap label 216B each include indicia 220 printed on the first, non-adhesive side 224 thereof while the vessel label 216C includes indicia 220 printed on the first side 224 and the second side 228 (see FIG. 15).

The kit 10 may also include one or more accessories 30 to be used in combination with the collection device 22 and/or swab 18 to help aid the collection and storage of the biological sample. Depending on the specific requirements of a particular sample, each kit 10 may be custom tailored so that the required accessories 30 are placed in the storage volume 42 before the kit 10 is sent to the patient.

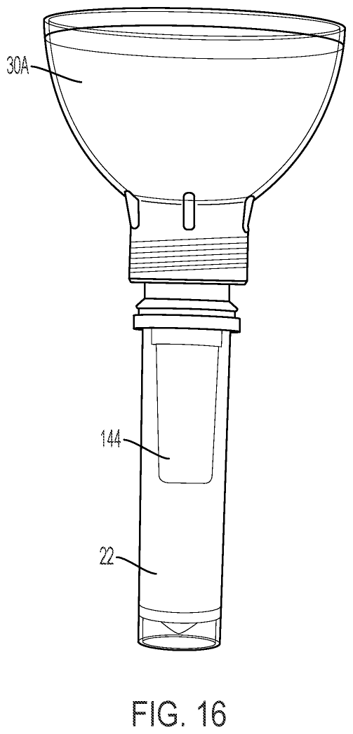

As shown in FIG. 1, one accessory may include a funnel 30A. The funnel 30A is sized and shaped to help a user spit or otherwise pour biological material directly into the vessel 144. In the illustrated embodiment, the funnel 30A includes a set of internal threads that are configured to correspond with the external threads of the first end 128 of the vessel 144 (see FIG. 16). As such, the funnel 30A may be connected to the vessel 144 in place of the cover 148 when the sample is being collected and then swapped out so the cover 148 is attached to secure the sample inside the volume 166 once collection is complete.

In addition to the funnel 30A, other accessories for the kit 10 may include cool or hot packs to regulate the temperature of the sample as it travels within the container 14, alternative sample collection systems (e.g., syringes, and the like), and/or spare or duplicate components when needed.

To use the container 14 for an in-home test, the container 14 generally begins with the cover 50 in the open position, both the first and second couplers 90a, 90b in deactivated conditions, and the partition 104 in the open position (see FIG. 3). With the partition 104 in the open position, the user has access the second portion 112 of the volume 42 via the opening 46. As such, the user may first load the desired components into the second portion 112 of the volume 42 via the opening 46 (e.g., the swab 18, the identification elements 26, the funnel 30A, any other accessories, and the like).

With the desired items loaded in the second portion 112, the user may then move the partition 104 into the closed position (see FIG. 4) thereby isolating the second portion 112 from the opening 42 and presenting the first portion 108 of the volume 42 to the opening 46. Placing the partition 112 into the closed position also properly orients the vessel support 124 within the volume 42.

With the partition 104 in place, the user may then insert the sample collection device 22 into the vessel support 124 such that the first end 128 is positioned in the first portion 108 of the volume 42 proximate the opening 46. (See FIG. 5.) With the items loaded, the user may then place the container 14 in the first secured closed condition. To do so, the user activates the second coupler 90b by removing the adhesive backing from the second connector 94b and then moves the cover 50 into the closed position. By doing so, the tab 98 and connector 94b come into contact with the body 36 and adhere thereto securing the cover 50 in the closed position. (See FIG. 6.) With the container 14 in the first secured closed condition, the container 14 and corresponding items placed therein can be securely mailed or otherwise delivered to the user at a remote location (e.g., at home, at a pharmacy, at a doctor's office, and the like). During travel, the volume 42 may not be accessed without damaging, modifying, and/or marking the cover 14 in such a way so as to be easily identified by the patient.

Upon arrival, the patient may then access the items within the container 14 by detaching the tab 98 from the cover 50 by pulling or otherwise releasing the releasable joint 100. Once detached, the user is then free to move the cover 50 back into the open position and therefore gains access to the first portion 108 of the volume 42. (See FIG. 7). The tab 98 remains permanently coupled to the body 36.

With the cover 50 opened, the patient is first presented with the cover 148 of the sample collection device 22. The patient may remove the sample collection device 22 from the volume 42 and place it to the side. The patient then subsequently moves the partition 104 into the open position whereby the patient can access the second portion 112 of the volume 42 and remove the elements contained therein (e.g., the swab 18, instructions, identification elements 26, and funnel 30A).

With the components removed and accessible, the patient may then begin the sample collection process. In one embodiment, the process may include 1) removing the cover 148 from vessel 144, 2) removing the swab 18 from its pouch (when present), 3) swabbing the desired area with the swab 18 to collect biological material, 4) depositing the collected material into the volume 166 of the vessel 144, and 4) returning the cover 148 to the vessel 144 to seal the volume 166 with the sample therein. In some embodiments, the user may attach the funnel 30A to the vessel 144 instead of using the swab to allow the user to spit directly into the vessel 144. In still other embodiments, the user may continue to add biological material into the volume 166 of the vessel 144 until the indicia 186 becomes obscured and can no longer be easily identified by the patient.

With the sample secured within the volume 166 of the vessel 144 by the cover 148, the patient may then prepare the kit 10 for return to the lab. To do so, the patient may first return the partition 104 to the closed position and then insert the sample collection device 22 into the vessel support 124 such that the first end 128 is positioned in the first portion 108 of the volume 42 proximate the opening 46. (see FIG. 7).

With the sample collection device 22 in position, the user may then place the container 14 in a second secured closed condition by activating the first coupler 90a by removing the adhesive backing from the first connector 94a and then moving the cover 50 into the closed position. By doing so, the connector 94a comes into contact with the body 36 and adheres thereto—securing the cover 50 in the closed position. (See FIG. 8.)

With the container 14 in the second secured closed condition, the container 14 and sample collection device 22 can be securely mailed or otherwise delivered to a lab or other facility for testing and analysis. During travel, the volume 42 may not be accessed without damaging, modifying, and/or marking the cover 14 in such a way so as to be easily identified by the patient.

When the container 14 arrives at the lab, the testing personnel may then open the cover 50 by tearing or otherwise cutting open the cover 50. Once complete, the testing personnel have access to the sample collection device 20 and the biological sample contained therein.

Clause 1. A storage container for storing a sample collection device, the storage container comprising, a body defining a first storage volume, wherein the first storage volume includes a first opening through which the first storage volume may be accessed, a cover coupled to the body, wherein the cover is adjustable between a closed position, in which the cover restricts access to the first storage volume via the first opening, and an open position, in which the cover does not restrict access to the first storage volume via the first opening, a first securing element coupled to the cover and configured to retain the cover in the closed position, and a second securing element coupled to the cover and configured to retain the cover in the closed position independent of the first securing element, wherein the second securing element is a one-time use device, wherein the second securing device interacts with the cover at a first point, and wherein the first point can be detached from the remainder of the cover.

Clause 2. The storage container of clause 1, wherein at least one of the first securing element and the second securing element include adhesive.

Clause 3. The storage container of clause 1, wherein the first securing element is a one-time use device.

Clause 4. The storage container of clause 1, wherein the cover is integrally formed with the body.

Clause 5. The storage container of clause 1, wherein the cover includes, a first portion configured to selectively obstruct access to the first storage volume via the first opening, a second portion extending from the first portion, wherein the first securing element interacts with the cover via the second portion, and a third portion extending from the second portion opposite the first portion, wherein the second securing element interacts with the cover via the third portion.

Clause 6. The storage container of clause 5, wherein the third portion is detachable from the second portion.

Clause 7. The storage container of clause 5, wherein the third portion is connected to the second portion via a perforated connector.

Clause 8. The storage container of clause 1, wherein the first storage volume is sub-divided by a wall into a first storage volume portion and a second storage volume portion, and wherein the wall is movable between a first position, in which the first storage volume portion is accessible via the first opening and the second storage volume portion is not accessible via the first opening, and a second position, in which both the first storage volume portion and the second storage volume portion are accessible via the first opening.

Clause 9. The storage container of clause 8, wherein the wall defines an aperture sized to receive at least a portion of the sample collection device therein.

Clause 10. A storage container comprising, a body defining a storage volume, wherein the storage volume includes a first opening through which the first storage volume may be accessed, a cover, wherein the cover is adjustable between a closed position, in which the cover restricts access to the first storage volume via the first opening, and an open position, in which the cover does not restrict access to the first storage volume via the first opening, wherein the cover includes a first portion, and a second portion extending from the first portion, a first securing element coupled to the first portion of the cover and configured to retain the cover in the closed position, and a second securing element coupled to the second portion of the cover and configured to retain the cover in the closed position independent of the first securing element.

Clause 11. The storage container of clause 10, wherein the second securing member is a one-time use device.

Clause 12. The storage container of clause 10, wherein the second securing member is an adhesive.

Clause 13. The storage container of clause 10, wherein the cover is formed integrally with the body.

Clause 14. The storage container of clause 10, wherein the cover further includes a third portion adjustably coupled to the body and configured to at least partially obstruct the first opening, and wherein the first portion extends from the third portion.

Clause 15. The storage container of clause 10, wherein the second portion is detachable from the first portion.

Clause 16. The storage container of clause 10, wherein the second portion is coupled to the first portion by a perforated strip.

Clause 17. A method of transporting and collecting a secure sample in a sample collection device, the method comprising, providing a storage container defining a first storage volume accessible via a first opening, the storage container including a cover adjustable between a closed position, in which the first storage volume is not accessible via the first opening, and an open position, in which the first storage volume is accessible via the first opening, the storage container also including a first securing element and a second securing element, placing an empty sample collection device into the first storage volume via the first opening, securing the cover in the closed position with the first securing element, opening the storage container by detaching the portion of the cover interacting with the first securing element and placing the cover in the open position, removing the empty sample collection device from the first storage volume and filling it with a sample, returning the filled sample collection device to the first storage volume, securing the cover in the closed position with the second securing element.

Clause 18. The method of clause 17, wherein at least one of the first securing element and the second securing element are a one-time use device.

Clause 19. A sample collection device comprising, a body defining a sample collection volume therein, wherein at least a portion of the body is transparent, and indicia visible along a sight line that passes through the sample collection volume.

Clause 20. The sample collection device of clause 19, further comprising a pre-determined volume of buffer in the sample collection volume, and wherein the sight line passes through the buffer when the sample collection device is in an upright orientation.

Clause 21. The sample collection device of clause 20, wherein the buffer has a first state where no sample is dissolved therein, wherein the buffer has a first opacity in the first state, wherein the buffer has a second state where desired volume of sample is dissolved therein, wherein the buffer has a second opacity greater than the first opacity in the second state.

Clause 22. The sample collection device of clause 19, wherein the sight line defines a sight length, and wherein the sight length is such that the indicia is not visible when the buffer is in the second state.

Clause 23. The sample collection device of clause 19, further comprising a label applied to the exterior of the body, wherein the label has a first side facing away from the body and a second side opposite the first side facing toward the body, and wherein the indicia is formed on the second side.

Clause 24. The sample collection device of clause 23, wherein the indicia is printed onto the second side of the label.

Clause 25. The sample collection device of clause 19, wherein the indicia includes black symbol printed on a white background.

Clause 26. The sample collection device of clause 23, wherein the label is applied to the exterior of the body to produce a covered portion and a window portion.

Clause 27. The sample collection device of clause 26, wherein the sight line passes through the window portion.

Clause 28. A sample collection device comprising, a body defining a sample collection volume therein, wherein at least a portion of the body is transparent, and a label applied to the exterior of the body, the label having an exterior surface facing away from the body and an interior surface facing toward the body, and indicia applied to the interior surface of the label.

Clause 29. The sample collection device of clause 28, wherein the indicia is visible through the body.

Clause 30. The sample collection device of clause 28, further comprising a pre-determined volume of buffer positioned within the sample collection volume.

Clause 31. The sample collection device of clause 30, wherein the indicia is visible through the buffer.

Clause 32. The sample collection device of clause 28, wherein the label extends a portion of the distance around the perimeter to define a gap, and wherein the indicia is at least partially aligned with the gap.

Clause 33. The sample collection device of clause 32, wherein the indicia is opposite the gap.

Clause 34. A kit for collecting and storing a biological sample, the kit comprising, a sample collection device defining a sample collection volume and a first open end, a cap configured to be removably coupled to the first open end to enclose the sample collection volume, a swab enclosed in first package, a storage container sized to receive the sample collection device, the cap, and the swab therein, and a set of labels configured to be applied to at least one of the sample collection device, the cap, the first package, the swab, and the storage container, and wherein each label of the set of labels contains matching identification information thereon.

Clause 35. The kit of clause 34, further comprising a funnel having a first end configured to be removably coupled to the first open end of the sample collection device and a second end opposite the first end, and wherein the second end is larger than the first end.

Clause 36. The kit of clause 34, wherein each label of the set of labels has a first surface upon which the identifying information is applied, and a second surface opposite the first surface where an adhesive is applied.

Clause 37. The kit of clause 36, wherein at least one label of the set of labels further includes secondary indicia applied to the second side.

Clause 38. The kit of clause 37, wherein the at least one label is applied to the sample collection device.

Clause 39. The kit of clause 34, wherein the identifying information may include any one of a QR code, a bar code, and alpha-numeric text.

Clause 40. The kit of clause 34, wherein the storage container defines a storage volume accessible via a first inlet, wherein the storage container includes a barrier at least partially positioned within the storage volume and configured to sub-divide the storage volume into a first volume portion and a second volume portion, and wherein the second volume portion is sized to receive the swab therein.

Clause 41. The kit of clause 40, wherein the barrier is adjustable between a first position, in which the first volume portion is accessible via the inlet and the second volume portion is not accessible via the inlet, and a second position, in which both the first volume portion and the second volume portion are accessible via the inlet.

Clause 42. The kit of clause 40, wherein the barrier defines an aperture therein sized to receive at least a portion of the sample collection device therein.

Clause 43. The kit of clause 42, wherein the aperture is sized and shaped so that the open end of the sample collection device is accessible via the inlet when the sample collection device is positioned within the aperture.

Clause 44. A kit for collecting and storing a biological sample, the kit including, a sample collection device defining a sample collection volume and a first open end, wherein the first sample collection device has a first indicia, a cap configured to be removably coupled to the first open end to enclose the sample collection volume, wherein the cap includes a second indicia, a swab enclosed in first package, and wherein one of the swab and the first package includes a third indicia, a storage container sized to receive at least one of the sample collection device, the cap, and the swab therein, and wherein the storage container includes a fourth indicia; and wherein the first indicia, the second indicia, the third indicia, and the fourth indicia all include matching identification information.

Clause 45. The kit of clause 44, wherein each indicia includes a sticker applied to its respective element.

Clause 46. The kit of clause 44, wherein the storage container defines a storage volume accessible via an inlet, and wherein the storage volume is sized to receive the sample collection device, the cap, and the swab therein.

Clause 47. The kit of clause 46, wherein the storage container further includes a barrier configured to separate the storage volume into a first portion and a second portion, and wherein the second portion is sized to receive the swab therein.

Clause 48. The kit of clause 47, wherein the barrier defines an aperture sized to at least partially receive the sample collection device therein, and wherein storage container is configured such that the first open end of the sample collection device is positioned within the first portion when the sample collection device is positioned within the aperture.

Clause 49. The kit of clause 47, wherein the barrier is adjustable between a first position, in which the first portion is accessible via the inlet and the second portion is not accessible via the inlet, and a second position, in which both the first portion and the second portion are accessible via the inlet.

Clause 50. The kit of clause 49, wherein the barrier defines an aperture sized to at least partially receive the sample collection device therein, and wherein storage container is configured such that the first open end of the sample collection device is positioned within the first portion when the sample collection device is positioned within the aperture.

Clause 51. The kit of clause 47, further comprising a funnel having a first end configured to be removably coupled to the first open end of the sample collection device and a second end opposite the first end, and wherein the second end is larger than the first end.

Clause 52. The kit of clause 44, wherein the first open end of the sample collection device includes external threads formed thereon.

1-18. (canceled)

19. A sample collection device comprising:
a body defining a sample collection volume therein, wherein at least a portion of the body is transparent; and
indicia visible along a sight line that passes through the sample collection volume.

20. The sample collection device of claim 19, further comprising a pre-determined volume of buffer in the sample collection volume, and wherein the sight line passes through the buffer when the sample collection device is in an upright orientation.

21. The sample collection device of claim 19, further comprising a first fluid in the sample collection volume, wherein the first fluid has a first state where no sample is dissolved therein, wherein the first fluid has a first opacity in the first state, wherein the first fluid has a second state where a desired volume of sample is dissolved therein, and wherein the first fluid has a second opacity greater than the first opacity in the second state.

22. The sample collection device of claim 21, wherein the sight line defines a sight length passing through the collection volume, and wherein the sight length is such that the indicia is not obscured when the first fluid is in the first state, and wherein the first fluid is obscured when the first fluid is in the second state.

23. The sample collection device of claim 19, further comprising a label applied to the exterior of the body, wherein the label has a first side facing away from the body and a second side opposite the first side facing toward the body, and wherein the indicia is formed on the second side.

24. The sample collection device of claim 23, wherein the indicia is printed onto the second side of the label.

25. The sample collection device of claim 19, wherein the indicia includes black symbol printed on a white background.

26. The sample collection device of claim 23, wherein the label is applied to the exterior of the body to produce a covered portion and a window portion.

27. The sample collection device of claim 26, wherein the sight line passes through the window portion.

28. A sample collection device comprising:
a body defining a sample collection volume therein, wherein at least a portion of the body is transparent; and
a label applied to the exterior of the body, the label having an exterior surface facing away from the body and an interior surface facing toward the body; and
indicia applied to the interior surface of the label.

29. The sample collection device of claim 28, wherein the indicia is visible through the body.

30. The sample collection device of claim 28, further comprising a pre-determined volume of buffer positioned within the sample collection volume.

31. The sample collection device of claim 30, wherein the indicia is visible through the buffer.

32. The sample collection device of claim 28, wherein the label extends a portion of the distance around the perimeter to define a gap, and wherein a sight line to view the indicia passes through the gap.

33. The sample collection device of claim 32, wherein the indicia is opposite the gap.

34-52. (canceled)

53. The sample collection device of claim 21, wherein the indicia includes a symbol printed on a background, and wherein the contrast between the symbol and the background is configured so that the indicia is not obscured when the first fluid is in the first state and is obscured when the first fluid is in the second state.

54. The sample collection device of claim 53, wherein the sight line defines a sight length passing through the collection volume, and wherein the sight length is sized so that the indicia is not obscured when the first fluid is in the first state, and wherein the first fluid is obscured when the first fluid is in the second state.

55. The sample collection device of claim 19, wherein the indicia is incorporated into the body.

56. The sample collection device of claim 21, wherein the first liquid has a opacity of 0 in the first state.
